(19) United States
(12) Patent Application Publication
(10) Pub. No.: US 2002/0068065 A1
(43) Pub. Date: Jun. 6, 2002

(54) Pharmaceutical composition having specific water activity

(76) Inventors: Ondrej Hendl, Portage, MI (US); Nancy J. Britten, Portage, MI (US)

Correspondence Address: Andrew M. Solomon, Pharmacia & Upjohn Company, Global Intellectual Property, 301 Henrietta Street, Kalamazoo, MI, 49001, US

(21) Appl. No.: 09/949,171

(22) Filed: Sep. 7, 2001

Related U.S. Application Data
(60) Provisional application No. 60/231,868, filed on Sep. 12, 2000.

(51) Int. Cl. A61K 39/38; A61K 39/12; A61K 39/00; A61K 31/545

(52) U.S. Cl. 424/184.1; 424/204.1; 424/234.1; 514/206

(57) ABSTRACT

A composition comprising:

(a) one or more bioactive agents; and

(b) a non-aqueous liquid carrier;

wherein immediately after manufacture of the composition, said composition can be administered to a host such that the one or more bioactive agents is released to the host and wherein said composition has been adjusted to have a water activity of between about 0.2 and about 0.5 is provided.

### CROSS-REFERENCE TO RELATED APPLICATIONS

This application claims the benefit of US provisional application Serial No. 60/231,868, filed Sep. 12, 2000, under USC § 119(e)(I).

### BACKGROUND OF THE INVENTION

1. Field of the Invention

The present invention relates to pharmaceutical compositions. More specifically, it relates to the use of more chemically stable pharmaceutical formulations comprising one or more bioactive substance in a non-aqueous liquid carrier, where said improved chemical stability is achieved by the adjustment of formulation water activity from about 0.2 to about 0.5.

2. Background of the Invention

In the pharmaceutical arts, drug delivery is an element as significant as drug activity. Many drugs or bioactive agents with apparent in vitro activity fail at the clinical level due to the inability to prepare, store, or deliver the bioactive agent to the site of action in effective concentrations over a sufficient period of time.

A vehicle for the stable storage and effective delivery profile of a bioactive agent is of great utility. Those skilled in the art will understand that storage stability and effective delivery profile are, to some extent, specific to bioactive agents, the condition for which the bioactive agent is administered, and the presenting condition of the subject.

The chemical stability and the improvement of chemical stability of pharmaceutical preparations are of great importance to the drug industry. Of particular concern has been the chemical stability of formulations where the pharmaceutical carrier and/or a component of the composition is an oxidizable oil or contains ingredient(s) capable of undergoing lipid oxidation during formulation manufacture and/or product storage. It is well known that lipid oxidation in a pharmaceutical composition can lead to reduced chemical stability of the product and a shorter shelf life. In addition to the oxidative tendencies of many commonly used oils and lipid containing excipients at room temperature, the preparation of pharmaceutical compositions often requires the heating or administration of radiation to the oil vehicles and/or excipients to adequately dissolve ingredients in the carrier or to sterilize the formulation. This heat stressing or radiation exposure promotes even more rapid rates of lipid oxidation in the compositions, thus frequently causing a decline in chemical stability. In the past, various methods have been employed in an attempt to enhance the chemical stability of such systems, including the addition of antioxidants, various stabilizers, etc.

Sustained release or oil based preparations are considered in WO97/49402 (Vlaminck); WO94/00105 (Sabater); U.S. Pat. No. 4,297,353 (Hawkins); U.S. Pat. No. 5,019,395 (Mahjour); U.S. Pat. No. 5,739,159 (Wolf); U.S. Pat. No. 5,162,057 (Akiyama); WO96/20698 (Levy) the teachings of which are incorporated herein by reference. Also incorporated by reference is WO98/41207 (Brown) addressing subcutaneous (S.C.) administration of antibiotic into the ear of an animal.

U.S. Pat. No. 5,721,359 discloses the molecule crystalline ceftiofur free acid (CCFA), which is a cephalosporin antibiotic intended for use in mammals, and in particular food animals (e.g., cattle, sheep, goats and swine). The patent suggests that oil suspensions of CCFA can be produced for administration to food animals where the oils are vegetable oils. The oils as disclosed in the patent are intended to be used in their natural form. An advantage touted by this molecule over other antibiotics, particularly those in the ceftiofur family is the ability for CCFA to yield a sustained release pharmaceutical composition. It has now been discovered that the sustained release profile is not readily predictable and reproducible in immediate post-production product that uses natural vegetable oils

U.S. Pat. No. 5,736,151 “Antibiotic Oil Suspensions” describes the inclusion of small amounts of water in oil suspensions of active drug, such as ceftiofur hydrochloride. The resulting suspensions have improved resuspendability so less shaking is required before dosing.

A section of the book Lipid Oxidation by Edwin N. Frankel (pages 195-197) discusses the use of water activity as a predictor of food quality. The article states that water activity is a key factor affecting the lipid oxidation in foods during storage and describes how lipid oxidation in foods is high at low water activity (0.1-0.2) but decreases as water activity increases to a value of 0.2-0.4, then increases again above 0.4, reaching a maximum in the range of 0.6-0.8. Mechanisms to explain the effect of water activity on lipid oxidation in foods are suggested.

Despite the above teachings, there still exists a need in the art for pharmaceutical compositions that can be administered on a controlled release or immediate release basis and wherein the release performance is predictable and reproducible immediately after manufacture of the product, as well as a need for a means for improving the stability of formulations otherwise prone to lipid oxidation.

### BRIEF SUMMARY OF THE INVENTION

In accordance with the present invention a novel pharmaceutical composition that can be administered on a sustained release or immediate release basis and wherein the release performance is predictable immediately after manufacture of the product is provided. More specifically, formulations of this invention have water activity levels of from about 0.2 to about 0.5.

One embodiment of the invention provides a pharmaceutical composition comprising:

(a) one or more bioactive agents; and

(b) a non-aqueous liquid carrier;

wherein immediately after manufacture of the composition, said composition can be administered to a host such that the one or more bioactive agents are released to the host and wherein said composition has been prepared such that the water activity of the composition is between about 0.2 and about 0.5.

In preferred embodiments, component (a) comprises CCFA and component (b) must include at least one of the following:

(1) a mixture of a modified or non-modified oxidizable oil with either a non-modified non-oxidizable oil or a non-modified non-oxidizing vehicle;

(2) a modified or non-modified oxidizable oil;

(3) a mixture of a non-modified or oxidizable oil with either a modified non-oxidizable oil or a modified non-oxidizing vehicle;

(4) a modified or non-modified non-oxidizable oil or non-oxidizing vehicle;

(5) a modified or non-modified non-oil;

(6) a vehicle containing oxidizable lipid components.

In particularly preferred embodiments component (b) is a mixture of modified oxidizable oil with a natural fully non-oxidizable oil, and even more preferably a mixture of modified cottonseed oil with saturated coconut oil.

Another embodiment of the present invention provides a method for producing a pharmaceutical composition comprising the steps of combining a non-aqueous liquid carrier with a bioactive substance and adjusting the resulting composition such that it has a formulation water activity of between 0.2 to about 0.5. The adjustment comprises the addition of water either prior to, during or after the combining step, and/or manufacturing the formulation under conditions of high humidity, and/or selecting hydrated bioactive subtances, vehicles, and/or excipients containing water—such that the water activity of the final formulation is about 0.2 to about 0.5. In preferred embodiments, the liquid carrier is additionally modified by the use of chemical, physical or mechanical means to produce a carrier that has a higher level of oxidation products as compared to its original, or non-modified form. Particularly preferred embodiments comprise the use of a combination of heat and gamma radiation. In addition, the modification step of this process may occur either prior to, after or both prior to and after the combining step.

A more specific aspect of this method comprises the steps of:

(a) heating natural cottonseed oil to increase its oxidation products and yield a modified cottonseed oil;

(b) combining said modified cottonseed oil with saturated coconut oil or saturated coconut oil products to yield a carrier vehicle; and

(c) adding crystalline ceftiofur free acid to said carrier vehicle;

(d) adjusting the water activity of the resulting composition to between about 0.2 and about 0.5 and, optionally, thereafter;

(e) heating said pharmaceutical composition;

(f) cooling said composition;

(g) filling one or more vials with said composition; and

(h) exposing said one or more vials to gamma radiation.

A further embodiment of the present invention provides the composition of the present invention for use in medical treatment.

An additional embodiment of the present invention provides the use of the inventive composition to prepare a medicament for treating or preventing a disease in a mammal.

A final embodiment of the present invention provides a method of treating or preventing a disease comprising administering to a mammal in need of such treatment an effective amount of the inventive composition. A preferred aspect of this invention is to treat bacterial infections in food animals or companion animals with an inventive CCFA composition.

An objective of the present invention is to provide more chemically stable pharmaceutical formulations where such formulations would otherwise be subject to undesirable effects from lipid oxidation.

Another object of the present invention is to provide a novel sustained release composition.

Still another object of the present invention is to provide a method for producing a novel sustained release composition.

An additional objective of the present invention is to provide a novel immediate release composition.

Yet another object of the present invention is to provide a method for producing a novel immediate release composition.

A further object of the present invention is to provide a method for treating a disease or condition in a mammal.

These, and other objects, will readily be apparent to those skilled in the art as reference is made to the drawings and detailed description of the preferred embodiment.

### DETAILED DESCRIPTION OF THE INVENTION

In describing the preferred embodiment, certain terminology will be utilized for the sake of clarity. Such terminology is intended to encompass the recited embodiment, as well as all technical equivalents which operate in a similar manner for a similar purpose to achieve a similar result.

1. Terminology Definitions

This invention will be better understood with reference to the following definitions:

“Bioactive substances” shall be broadly understood to mean pharmaceuticals, immunogenic and immunomodulator compositions (including adjuvants), vectors such as liposomes and live vectors such as plasmids, viruses, prions, spores, nutritional supplements and bacteria and mixtures thereof.

These include, but are not limited to nutritional supplements, anti-infectives (e.g., antibiotics, antifungals, anti-virals), antineoplastics (e.g., anticancer agents, such as cis-platinum compounds), immunomodulators (e.g., antihistamines, immunoenhancers and immunosupressors), laxatives, vitamins, decongestants, gastrointestinal sedatives, antacids, anti-inflammatory substances (e.g., selective cox-2 inhibitors), anti-manics, vasodilators (coronary, cerebral and peripheral), psychotropics, stimulants, anti-diarrheal preparations, anti-anginal drugs, analgesics, anti-pyretics, hypnotics, sedatives, anti-emetics, growth promoters, anti-nauseants, anti-convulsants, neuro-muscular drugs, hyper and hypo glycemic agents, thyroid and anti-thyroid preparations, diuretics, cytotoxic compounds, anti-spasmodics, antiarthritics, uterine relaxants, anti-obesity drugs, anthelmintics, hormones, vaccines, mineral and nutritional additives, CNS agents not disclosed in this listing and any mixtures thereof. Specifically preferred bioactive agents include, but are not limited to, crystalline ceftiofur free acid (CCFA), platinum compounds (e.g., cis-platinum), ibuprofen, piroxicam, 1-[2-(4-fluorobenzoyl)aminoethyl]-4-(7-methoxynaphthyl) piperazine hydrochloride (FAMP), camptothecin, paclitaxel, flucytosine, and quinine.

“Sustained delivery or Sustained release” as used in relation to bioactive substances shall mean continued release or distribution of the bioactive substance such that the amount of bioactive remains in the patient's blood levels at a concentration of greater than a certain value (that value being one that produces therapeutically effective blood levels of active substance) over an extended period of time. The effective sustained release blood levels desired would, of course, differ depending on the bioactive substance, the disease being treated, the patient, and the like, is considered to be known to the skilled artisan and can be determined by routine experimentation. More specifically, a sustained delivery vehicle differs from an immediate delivery vehicle in that the immediate delivery vehicle releases its bioactive material at faster rate then the sustained delivery vehicle, potentially requiring more administrations of bioactive per treatment regimen. For example, if the bioactive substance is ceftiofur crystalline free acid (CCFA), the desired level of ceftiofur metabolites in the patient's blood plasma is noted to be about 0.2 μg/ml. In one embodiment of the invention, a single dose of sustaining-vehicle CCFA maintains a ceftiofur metabolite level in the blood plasma of at or above about 0.2 μg/ml for at least three and preferably at least about four and more preferably at least about five days post-administration (sustained delivery of CCFA). Comparisons as to the degree of sustained delivery are made with equivalent bioactive agents. That is, sodium salts to sodium salts and free bases to free bases. Sustained delivery as used in this document is to be specifically reconciled with the regulatory definition for the same term that requires that the concentration versus time profile have three distinct phases (i.e., an increasing concentration phase, a plateau phase and a concentration depletion phase). While the term sustained delivery as used in this document may encompass the above regulatory definition it is not intended to be limited to it as compositions which are sustained delivery as defined herein need not possess the three distinct phases (e.g., the composition may have an increasing concentration phase and an extended concentration depletion phase).

“Liquid carriers” include triglyceride fats and oils, including those derived from vegetable, animal, and marine sources. The liquid carrier is non-aqueous and preferably comprises an oxidizable oil or a liquid vehicle containing oxidizable lipid components. The liquid carrier may be fully saturated, partially or fully unsaturated and may be deemed an “oil” (which may be naturally occurring or synthetic) or a “non-oil”. Examples of liquid carriers which are partially or fully unsaturated hydrocarbons include, but are not limited to naturally occurring oils such as castor oil, safflower oil, cottonseed oil, corn oil, olive oil, cod liver oil, almond oil, avocado oil, palm oil, sesame oil, peanut oil, calendula oil and soybean oil. By way of example cottonseed oil is available in a preparation of 70% unsaturated fatty acids (Sigma, St. Louis, Mo.). Examples of fully saturated liquid carriers include, but are not limited to, esters of medium to large chain fatty acids (e.g., fatty acid triglycerides with a chain length of about C6 to about C24). Mixtures of fatty acids are split from the natural oil (e.g., coconut oil, palm kernel oil, babassu oil, etc.) and are refined. In some embodiments, about C8 to about C12 fatty acid medium chain triglycerides (MCT) are useful. These saturated vehicles are comprised of capric acid (about 20 to about 45%) and caprylic acid (about 45 to about 80%). Other oils include, but are not limited to glyceride vehicles comprising a glycerol ester, or propylene glycol di-esters, of a saturated or/and unsaturated fatty acid. Fully saturated liquid carriers include, but are not limited to, saturated coconut oil (which typically includes a mixture of lauric, myristic, palmitic, capric and caprylic acids), including those sold under the MIGLYOL trademark from Huls and bearing trade designations 810, 812, 829 and 840. Also noted are the NeoBeeRTM products sold by Drew Chemicals. Isopropyl myristate is another example of a non-oxidizing vehicle of the current invention. Examples of synthetic oils include tri-glycerides, or propylene glycol di-esters of saturated or unsaturated fatty acids having from 6 to 24 carbon atoms. Such carboxylic acids are meant to comprise those carboxylic acids having from 6 to 24 carbon atoms such as, for example hexanoic acid, octanoic (caprylic), nonanoic (pelargonic), decanoic (capric), undecanoic, lauric, tridecanoic, tetradecanoic (myristic), pentadecanoic, hexadecanoic (palmitic), heptadecanoic, tetracosanoic, nonadecanoic, hexadecanoic (palmitic), heptadecanoic, ocatdecanoic (stearic) nonadecanoic, eicosanoic, heneicosanoic, docosanoic and lignoceric acid. Examples of unsaturated carboxylic acids include oleic, linoleic, linolenic acid and the like. It is understood that the tri-glyceride vehicle may include the mono-, di-, or triglyceryl ester of the fatty acids or mixed glycerides and/or propylene glycol di-esters wherein at least one molecule of glycerol has been esterified with fatty acids of varying carbon atom length, preferably carbon lengths of between about 6 to about 24 carbon atoms, more preferably between about 8 and about 18. Some non-limiting examples of “non-oils” include polyethylene glycol (PEG).

Oxidizable lipid components of water activity adjusted formulations of the present invention include any excipient capable of undergoing lipid oxidation. These oxidizable excipients may contain aliphatic unsaturated straight or substituted fatty acids with one or more double bonds. Phospholipons are a non-limiting example of such an excipient. If water activity adjusted formulations of the current invention contain one or more oxidizable lipid excipients, as described above, the pharmaceutical carrier may be comprised of an oxidizable oil vehicle, a non-oxidizable vehicle, or a combination of both oxidizable and non-oxidizable vehicles.

“Lipid” shall be broadly understood to mean any group of fats or fat like substances including fatty acids, neutral fats, waxes, and steroids; all contain aliphatic hydrocarbons, are water insoluble, and are easily stored in the body.

“Modified” and “modification” as to the vehicles of this invention and as used in the claims shall be understood to define a vehicle which, through physical, chemical or mechanical means, has been altered as compared to its natural (or “non-modified” in the case of synthetic liquid carriers) form such that the modified vehicle has an increased level of oxidation products and, when combined with the bioactive substance, yields a composition having a water activity of between about 0.2 and about 0.5. Modification can be accomplished by heat modification, irradiation (e.g., gamma or microwave radiation), addition of catalysts (i.e., t-butyl peroxide), the incorporation of specific triglycerides and their hydroperoxides, incorporation of polymeric species, incorporation of crosslinkers or of polymerization causing agents, oxidation regimens and combinations of these methods. These steps can be taken before or after addition of the drug to the vehicle, or both before and after addition of drug to the vehicle. In accordance with preferred embodiments of the present invention, the modification takes place in connection with the pharmaceutically acceptable liquid carriers which are either partially or fully unsaturated, although it is specifically contemplated that modification of the fully saturated liquid carriers or non-oils is also possible. Where modification relates to adjusting the water activity of the resulting composition, the adjustment can take place via the addition of water to the ultimate composition either before, during or after the combining of the bioactive substance with the modified liquid carrier, and/or conducting any of the processing steps under conditions of high environmental humidity, and/or by selecting hydrated bioactive substances, vehicles, and/or excipients containing water—such that the water activity of the resulting final formulation is from about 0.2 to about 0.5. Moreover, the liquid carrier, per se, need not be specifically modified to a given water activity; it is the resulting composition which is modified as such.

“Substantially peroxidized oxidizable oil vehicle” shall refer to a modified liquid carrier having a peroxide value of between about 0.1 and about 600, and in some embodiments about 10, about 20, about 40, or about 80 or any value in between. As used herein, peroxide values are expressed as milliequivalents (mEq) of peroxide per 1000 grams of oil sample. Peroxide value is conveniently measured by American Oil Chemists' Society (AOCS) (Official Method Cd 8-53) (Official Monographs, Soybean Oil, page 1434) manual titration, the teachings of which are incorporated herein by reference.

“Water activity” (WA) in heterogeneous systems determines the distribution of water between different components (i.e. bioactive agent and oil phase or non-oil phase or their mixture). The WA is defined as WA=p/pₒ, where p=partial pressure of water in formulation, pₒ=vapor pressure of water. From the literature is known the effect of water activity on lipid oxidation in food. Lipid oxidation is high at low water activity (0.1-0.2) but decreases as water activity is increased to a minimum, up to 0.2-0.4, and then increases again above 0.4, reaching maximum in the range 0.6-0.8. In accordance with the present invention, the desired water activity of the inventive composition is between about 0.2 and about 0.5.

2. The Invention

The present invention comprises a composition comprising:

(a) one or more bioactive agents; and

(b) a non-aqueous liquid carrier;

wherein immediately after manufacture of the composition, said composition can be administered to a host such that the one or more bioactive agents is released to the host and wherein said composition has been adjusted to have a resulting water activity of between about 0.2 to about 0.5.

It is a substantial advantage to identify a dosage form and method of preparation of a dosage form that provides a desired release profile either a sustained release or immediate release immediately upon production and maintains that release profile during a substantial storage period. In the present invention, a combination of preparatory steps and vehicle compositions are defined which yield desired release formulations upon processing. Water activity adjusted formulations may be obtained by the use of, a modified or non-modified liquid carrier, which can be in the form of an oxidizable oil, a liquid vehicle containing oxidizable lipid components, a non-oxidizable oil, a non-oxidizing vehicle, a non-oil or any mixture thereof in combination with the bioactive agent. In certain embodiments where a modified liquid carrier is utilized, in addition to the modified component, the liquid carrier can also optionally include a non-modified oxidizable oil, a non-modified non-oxidizable oil, a non-modified non-oxidizing vehicle, a non-modified non-oil or any mixture thereof. In all preferred embodiments, a key feature is that of the formulation has been adjusted either before, after or both before and after it has been combined with the bioactive agent and that the resulting composition has been prepared to have a water activity ranging from about 0.2 to about 0.5.

The bioactive agents for use are as defined above. A preferred bioactive agent is crystalline ceftiofur free acid (CCFA) which is useful as an antibiotic drug compound in pharmaceutical dosage forms for treating valuable mammalian animals and humans suffering from bacterial infections. In particular embodiments, sustained release ceftiofur free acid is useful as a veterinary antibiotic drug to treat animals such as cattle, swine, horses, sheep, goats, dogs, poultry and cats. Such treatment fights the effects of bacterial infections caused by susceptible organisms, such as Pasteurella haemolyitica, Pasteurella multocida, Salmonella typhimurium, Salmonella choleraesuis, Actinobacillus pleuropneumoniae, Streptococcus suis, Haemophilus somnus, Escherichia coli, Staphylococcus aureus and the like, as well as applicable anaerobic infections. These types of infections are commonly associated with diseases in animals, such as bovine respiratory disease and swine respiratory disease.

In specific embodiments, the following are used as the carrier vehicles of the instant invention:

(1) a mixture of a modified or non-modified oxidizable oil with either a non-modified non-oxidizable oil or a non-modified non-oxidizing vehicle;

(2) a modified or non-modified oxidizable oil;

(3) a mixture of a non-modified or oxidizable oil with either a modified non-oxidizable oil or a modified non-oxidizing vehicle;

(4) a modified or non-modified non-oxidizable oil or non-oxidizing vehicle;

(5) a modified or non-modified non-oil; or

(6) a vehicle containing oxidizable lipid components.

Other combinations which include formulations with water activities of from about 0.2 to about 0.5 can be envisioned by one skilled in the art and are expressly covered as being within the scope of the instant invention.

A preferred embodiment of the present invention is where the delivery vehicle is the combination of a modified oxidizable oil with a fully non-oxidizable oil or nonoxidizing vehicle. In even more preferred embodiments, the modified oxidizable oil is a substantially peroxidized oxidizable oil vehicle. For this embodiment, the ratio of modified oxidizable oil to non-oxidizable, non-oxidizing oil is from about from about 0.01:99.99 to about 90:10 (v/v), the total amount of each being 100 percent, with particular reference to the range from about 10:90 to about 25:75 (v/v), and most particularly from about 10:90 to about 20:80 (v/v).

An example of this is if the modified oxidizable oil comprises modified cottonseed oil and the non-modified non-oxidizable oil or non-modified non-oxidizing vehicle comprises saturated coconut oil or a saturated coconut oil product (for example MIGLYOL 812). So called “induced” cottonseed oil which has a higher level of oxidation products as a result of natural cottonseed oil having been heated and/or irradiated in the presence of oxygen is specifically contemplated as being a type of modified cottonseed oil. When the bioactive agent is CCFA, it is preferably combined with this example vehicle such that the concentration of the CCFA in the composition ranges between about 50 mg/ml to about 250 mg/ml and more preferably between about 100 mg/ml to about 200 mg/ml.

A useful processing scheme for producing a sustained release product of this embodiment is described below. Natural (non-modified) cottonseed oil is added to a mixing tank which is then heated and sparged with air to increase the peroxide value. The cottonseed oil is then cooled and sparged with nitrogen. The cottonseed oil at this point is deemed modified cottonseed oil. The vehicle is then prepared by mixing 20 parts by volume of modified cottonseed oil with 80 parts by volume of a saturated coconut oil or saturated coconut oil product, for example Miglyol 812. Drug (bioactive substance, for example CCFA) is added to the vehicle and the mixture is purged with nitrogen. The purged mixture is heated and the release rate of the drug is monitored using an in process assay procedure to determine when the desired release rate is achieved. At this point the heating is terminated and the mixture is cooled, the water activity of the final formulation is adjusted to from about 0.2 to about 0.5, suspension is filled into vials and terminally sterilized by gamma irradiation and released against final specifications.

Revisiting the above process, it is contemplated that desired release formulations of other embodiments can be achieved by alternate routes within the disclosed process. For example, in one such process, drug is added to a non-modified oxidizable oil and directly subjected to terminal irradiation to modify the oxidizable oil and produce a sustained release vehicle. In another, the process is terminated after fill and without terminal sterilization. In an embodiment consisting of a modified non-oil such as PEG 400, the drug/PEG-400 mixture is purged with nitrogen, heated, cooled and filled. It is an important aspect of the invention that not all processing steps are required to result in a sustained release preparation in every protocol. However, in accordance with the present invention adjustment of the water activity of the final formulation to a range of from about 0.2 to about 0.5 is required.

In addition, the composition is adjusted either before, during or after combination of the bioactive substance with the non-aqueous liquid carrier such that the water activity of the resulting composition is between about 0.2 and about 0.5. The adjustment of water activity levels for formulations of the current invention may be accomplished by the addition of water to the formulation before, during, and/or after incorporation of the bioactive substance, and/or may be achieved by manufacturing and/or filling of the formulation under intentionally high environmental humidity levels, and/or may be achieved by selection of hydrated bioactive substances, vehicles, and/or excipients containing water—such that the resulting final formulation has a water activity of from about 0.2 to about 0.5. The water activity levels for formulations of the instant invention are the result of water incorporated directly into the formulation, as well as water contributed by the bulk bioactive substance(s), by the pharmaceutical carrier, by any excipients added, and by the processing conditions. The formulation water activity level is also dependent upon the physical and chemical properties of the vehicle used to prepare the composition, on the physical/chemical properties and concentration of the bioactive substance(s) and excipients incorporated, as well as upon the physical/chemical properties of each resulting individual final formulation (i.e., the effect of the interaction of all of the above components combined in the final formulation, on the partial pressure of water in the formulation).

The water activity of a formulation comprised of bioactive substance(s) and oxidizable lipid component(s), is a more appropriate and useful physicochemical property to measure and control, than is water content—since water activity is the thermodynamic availability of water in the system rather than just the amount of water present. The relationship between water activity and water content will vary, sometimes considerably, depending upon the specific pharmaceutical formulation—since the partial pressure of water in the formulation (the most variable factor of water activity) is dependent on the interaction of a number of variables in the final formulation (see the preceding paragraph). Since the formulation water activity is a dynamic/interactive property, while water content in a formulation is much more static—it is easy to see why the relationship between formulation water activity and water content will be different for each specific formulation. Since the means for the empirical determination of the relationship between formulation water activity and water content of a formulation are not currently available, it is necessary to experimentally generate data relating these two properties for each individual pharmaceutical formulation.

In addition to the instant inventive vehicle of the instant invention, the compositions of this invention can be employed in admixture with conventional excipients, i.e., pharmaceutically acceptable organic or inorganic carrier substances suitable for parenteral, enteral (e.g., oral or inhalation) or topical application which do not deleteriously react with the active compositions. Suitable pharmaceutically acceptable carriers include but are not limited to water, salt solutions, alcohols, gum arabic, vegetable oils, benzyl alcohols, polyethylene glycols, gelatine, carbohydrates such as lactose, amylose or starch, magnesium stearate, talc, silicic acid, viscous paraffin, perfume oil, fatty acid esters, hydroxy methylcellulose, polyvinyl pyrrolidone, etc. The pharmaceutical preparations can be sterilized and if desired mixed with auxiliary agents, e.g., lubricants, preservatives, stabilizers, wetting agents, emulsifiers, salts for influencing osmotic pressure, buffers, coloring, flavoring and/or aromatic substances and the like which do not deleteriously react with the active compositions. They can also be combined where desired with other active agents, e.g., vitamins. In specific embodiments, the liquid carrier may additionally contain a thickening agent, for example, beeswax, hard paraffin or cetyl alcohol. Also noted as optional additives are benzyl alcohols, polyethylene glycols, viscous paraffin, perfume oil, and fatty acid esters.

The inventive compositions are useful for human and veterinary medicine. More specifically, the compositions of the present invention can be used to treat humans, food animals or companion animals. This includes, but is not limited to the following: food animals such as cattle, swine, sheep, goats and deer; companion animals such as cats, birds, dogs, horses and fish; poultry; or humans. The amount of inventive composition to be administered is that which will deliver the bioactive agent in an amount and for a duration to provide a therapeutic benefit necessary to treat or prevent a disease without causing toxicity problems to the patient. The specific amounts to be selected are deemed to be within the skill of the artisan. For example, when CCFA is selected as the bioactive agent, it is administered in unit dosage form for intramuscular or subcutaneous administration comprising about 0.5 to about 10.0 mg CCFA/kg body weight of patient with preferred ranges of about 4.4-6.6 mg/kg for cattle, and 5.0-7.5 mg/kg for swine. To the extent necessary for completion the dosages as described in U.S. Pat. Nos. 5,721,359 and 6,074,657 are expressly incorporated by reference.

Administration of the composition is contemplated to include chronic, acute or intermittent regimens, and any mode where liquid administration is feasible may be selected. The compositions of the present invention can be administered parenterally (for example, by intravenous, intraperitoneal or intramuscular injection), topically (including but not limited to surface treatment, transdermal application, and nasal application), intravaginally, orally, or rectally.

For oral therapeutic administration, the composition may be administered in the form of capsules, elixirs, suspensions, syrups, and the like. Such compositions and preparations should, typically, contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active composition may be incorporated into sustained-release preparations and devices such as the osmotic release type devices developed by the Alza Corporation under the OROS trademark.

For parenteral application, the compositions can be administered intravenously or intraperitoneally, by infusion or injection. In one embodiment where CCFA is the bioactive agent, subcutaneous ear injection in accordance with U.S. Pat. No. 6,074,657 is an appropriate mode of administration. Intramuscular administration is also specifically contemplated.

For topical administration, the composition may be applied in the form of drops (for example to treat diseases or infections of the eye), or for skin application in the form of spreadable pastes, gels, ointments, soaps, and the like. The resultant liquid compositions can additionally be applied from absorbent pads or suppositories, used to impregnate bandages and other dressings, or sprayed onto the affected area using pump-type or aerosol sprayers.

It will be appreciated that the actual preferred amounts of active compositions in a specific case will vary according to the specific compositions being utilized, the particular compositions formulated, the mode of application, and the particular situs and organism being treated. Dosages for a given host can be determined using conventional considerations, e.g., by customary comparison of the differential activities of the subject compositions and of a known agent, e.g., by means of an appropriate, conventional pharmacological protocol.

In certain embodiments, an important aspect of the present invention is that by performing the modification to the carrier vehicle, the in vivo performance of the bioactive substance can be entirely controlled and predictable. To this extent, while not wishing to be bound to any particular scientific theory, the performance of the bioactive substance is “locked in” immediately post manufacture. As such, the performance of the bioactive substance in vivo is comparable (i.e., the active is released to the host such that it remains in the host at a therapeutically effective level for a desired period of time) from the time of manufacture for many months of storage time. As an example, the performance of the bioactive administered in vivo 30 days, 60 days, 90 days, 180 days, 360 days or 720 days after manufacture is comparable to the performance just after manufacture.

While the present invention is primarily directed to sustained release delivery vehicles, it is also expressly contemplated that for certain bioactive substances advantages can be obtained without the vehicle being a sustained release one. For example, the chemical or physical stability of the final formulation could be improved. Further for parenteral formulations the compositions of this invention may provide reduced injection site irritation for certain tissue irritating bioactive substances. For oral formulations compositions of this invention could possibly provide protection against stomach irritation by certain bioactive substances, may help mask the taste of poorly palatable drugs, and might be used to target delivery of certain drugs (i.e. where it's desired that absorption of the active medicament occur lower in the G.I. tract, rather than in the stomach—for greater efficacy, to target certain disease conditions, etc.) For intramammary formulations compositions of this invention could possibly provide reduced udder irritation, and might prevent or reduce systemic absorption of the drug from the udder, leaving more medicament at the site of the infection, thus improving efficacy and increasing chances for reduced slaughter times.

The monitoring of water activity in foods, the fact that specific food water activity levels correlate to certain lipid oxidation rates, and the maintenance of food stability by the use of protective packaging is known in the food industry. The current invention is the first time that this knowledge of the relationship between water activity and lipid oxidation has been applied to the preparation of more chemically stable pharmaceutical formulations by the novel method of adjusting the formulation's water activity level to about 0.2 to 0.5. Methods and compositions of the present invention add a significant new tool to the formulator's arsenal of means to control and improve the chemical stability of pharmaceutical preparations containing components capable of lipid oxidation.

The invention is further described in the following non-limiting examples.

### Preparation 1—Cottonseed Oil Modification

A substantially peroxidized oxidizable is prepared from natural cottonseed oil. 105 parts by volume of natural cottonseed oil are added to a vessel having a steam jacket for heating. Steam is applied to the jacket to heat the oil to between about 85 and about 110° C. Air is bubbled through the oil while it is agitated. The flow rate of the air varies from about 1 standard cubic foot per hour (SCFH)/liter to 20 SCFH/liter. Agitation is such that the temperature of the oil remains constant over the time period of heating. The oil is heated for a time and at a temperature necessary to achieve a peroxide value as measured by the method of the US Pharmacopea (USP 24 NF 19 at page 1870) or by AOCS method 8-53 and then cooled, transferred to a different container and stored under nitrogen conditions. To achieve a peroxide value of about 10, at a temperature of about 89° C. the oil is heated for about 9 hours, at a temperature of about 100° C. the oil is heated for about 3 hours, and at a temperature of about 105° C. the oil is heated for about 2.3 hours. To achieve a peroxide value of about 40, at a temperature of about 100° C. the oil is heated for about 6.75 hours, and at a temperature of about 105° C. the oil is heated for about 5.5 hours. To achieve a peroxide value of about 80, at a temperature of about 105° C. the oil is heated for about 8 hours. The relationship between the time and temperature of the oil as compared to its peroxide value is considered to be linear and one skilled in the art could achieve a desired peroxide value depending on the time and temperatures selected for processing.

### PREPARATION EXAMPLE 1

(i) 10 parts by volume of the modified cottonseed oil prepared according to Preparation 1 and having a peroxide value of between about 10-40 are mixed with 90 parts by volume of Miglyol 812 to form a carrier vehicle.

(ii) 0.1 parts by weight of CCFA are added and mixed for 1-3 hours to form a uniform suspension such that the concentration of CCFA is 100 mg/ml.

(iii) The suspension is heated to about 85-110° C. for about 2-20 hours and permitted to cool.

(iv) The suspension is packaged and sterilized with gamma radiation.

The resulting product is a stable, sustained release formulation of CCFA having a concentration of 100 mg/ml.

### PREPARATION EXAMPLE 2

The procedure of Preparation Example 1 is repeated except that in step (i) the ratio of modified cottonseed oil to Miglyol 812 is 20:80, and in step (ii) the amount of CCFA added is such that the concentration of CCFA is 200 mg/ml. The resulting product is a sustained release formulation of CCFA having a concentration of 200 mg/ml.

### PREPARATION EXAMPLE 3

The procedure of Preparation Example 2 is repeated except that steps (iii) and (iv) are replaced with the step of filling vials with the suspension and terminally heating the packaged suspension to 85-110° C. for about 3-14 hours and allowing the vials to cool. The resulting product is a stable, sustained release formulation of CCFA having a concentration of 200 mg/ml.

### PREPARATION EXAMPLE 4

The procedure of Preparation Example 3 is repeated except that the following step (i) is substituted for the identical step in Preparation Example 3 and in step (ii) the amount of CCFA added is such to obtain a final concentration of 100 mg/ml.

(i) Native peanut oil is modified by heating it to 90-100° C. for a period of 1-10 hours to yield a composition having a peroxide value of between 10-80. 1 part of the modified peanut oil by volume is mixed with 99 parts by volume of Miglyol 810 for 1-3 hours.

The resulting product is a stable, sustained release formulation of CCFA having a concentration of 100 mg/ml.

### PREPARATION EXAMPLE 5

A formulation is prepared as set forth in Preparation Example 1 except that the following step (i) is substituted for the identical step in Preparation Example 1, in step (ii) the amount of CCFA added is such to obtain a final concentration of 250 mg/ml, and step (iii) is omitted.

(i) Native corn oil is modified by heating it to 90-100° C. for a period of 1-10 hours to yield a composition having a peroxide value of between of 5-50. 5 parts by volume of this is mixed with 95 parts by volume of Miglyol 829 for 1-3 hours to yield a carrier vehicle.

### PREPARATION EXAMPLE 6

(i) 40 parts by volume of modified sesame oil prepared using the heat treatment according to Preparation 1 and having a peroxide value of between about 4-60 are mixed with 60 parts by volume of isopropyl myristate to form a carrier vehicle.

(ii) 0.15 parts by weight of ceftiofur crystalline free acid are added and mixed for 1-3 hours to form a uniform suspension such that the concentration of CCFA is 150 mg/ml.

(iii) The suspension is packaged and heated to 85-110° C. for about 3-14 hours and permitted to cool.

The resulting product is a stable, sustained release formulation of CCFA having a concentration of 150 mg/ml.

### PREPARATION EXAMPLE 7

(i) 15 parts by volume of safflower oil are mixed with 85 parts by volume of Miglyol 840 to form a carrier vehicle.

(ii) 0.30 parts by weight of CCFA are added and mixed for 1-3 hours to form a uniform suspension such that the concentration of CCFA is 300 mg/ml.

(iii) The suspension is packaged subjected to microwave radiation to modify the safflower oil and permitted to cool.

The resulting product is a stable, sustained release formulation of CCFA having a concentration of 300 mg/ml.

### PREPARATION EXAMPLE 8

(i) 100 parts by volume of palm oil are prepared using the heat treatment according to Preparation 1 to have a peroxide value of between about 10-100 to form a carrier vehicle.

(ii) 0.10 parts by weight of ceftiofur crystalline free acid are added and mixed for 1-3 hours to form a uniform suspension such that the concentration of CCFA is 100 mg/ml.

(iii) The suspension is heated to 85-110° C. for about 2-20 hours and permitted to cool.

(iv) The suspension is packaged and sterilized with gamma radiation.

The resulting product is a stable, sustained release formulation of CCFA having a concentration of 100 mg/ml.

### PREPARATION EXAMPLE 9

(i) 0.1 parts by weight of CCFA are added to 10 parts by weight of native cottonseed oil and mixed for 1-3 hours to form a uniform suspension such that the concentration of CCFA is 100 mg/ml.

(ii) The suspension is heated to about 85-110° C. for about 2-20 hours and permitted to cool.

(iii) The suspension is packaged and sterilized with gamma radiation.

### EXAMPLES 1-9

The water activity of the compositions of Preparation Examples 1-9 is adjusted such that the water activity of the resulting composition is between about 0.2 and about 0.5. The adjustment of water activity can be accomplished by the addition of water to the compositions either prior to, during, or after combination of the bioactive substance to the carrier liquid, and/or may be accomplished by either the direct addition of water or by the processing of the composition in a high humidity environment, and/or may be accomplished by the selection of hydrated bioactive substances, vehicles, and/or excipients containing water—such that the water activity of the final composition is from about 0.2 to about 0.5.

### EXAMPLE 10

A preparation of 50 mg/ml of Piroxicam in Corn Oil having an adjusted water activity of between 0.2 and about 0.5 is produced as follows:

Method 1:

Measure the concentration of water present in the bulk drug and in the Corn Oil used. Prepare several small scale lots of 50 mg/mL Piroxicam in Corn oil. To these lots add various concentrations of water for injection. Manufacture under conditions of 85% relative humidity. Measure the water activity (Aw) for each of the test formulations and determine the concentration of water that will be needed for the final Piroxicam formulation to achieve a water activity level of 0.2 and 0.5 (taking into account the concentrations of water present in the specific lot of bulk drug and lot of Corn oil to be used) and add such water to the formulation.

Method 2:

Add Corn oil NF to a manufacturing tank under conditions of 85% relative humidity. Add to the Corn oil the amount of water for injection, USP (determined in the section above) that will provide a final formulation water activity of 0.2 to about 0.5, and mix thoroughly. Add Piroxicam and mix adequately. Fill formulation into vials.

### Testing of Inventive Compositions

Cows suffering from either bovine respiratory disease or an anaerobic infection of the interdigital space are injected with any of the compositions of Examples 1-9 such that the level of administration of CCFA is between about 4.4 to about 6.6 mg CCFA/kg animal body weight. Administration is by subcutaneous injection in the neck or subcutaneous injection in the ear as described in U.S. Pat. No. 6,074,657. The concentration of effective CCFA metabolites in the blood plasma of the cows rises to at least 0.2 μg/ml within one hour of administration and remains at or above this level for at least 80 to about 140 hours. Only one administration of CCFA is required for the treatment regimen.

Having described the invention in detail and by reference to the preferred embodiments thereof, it will be apparent that modifications and variations are possible without departing from the scope of the appended claims.

What is claimed is:

1. A composition comprising:
(a) one or more bioactive agents; and
(b) a non-aqueous liquid carrier;
wherein immediately after manufacture of the composition, said composition can be administered to a host such that the one or more bioactive agents is released to the host and wherein said composition has been adjusted to have a water activity of between about 0.2 and about 0.5.

2. The composition according to claim 1 wherein said one or more bioactive agents is selected from the group consisting of pharmaceuticals, immunogenic and immunomodulator compositions, vectors, viruses, prions, spores, nutritional supplements and bacteria and mixtures thereof.

3. The composition according to claim 2 wherein said one or more bioactive agents is selected from the group consisting of nutritional supplements, anti-infectives, antineoplastics, immunomodulators, laxatives, vitamins, decongestants, gastrointestinal sedatives, antacids, anti-inflammatory substances, anti-manics, vasodilators (coronary, cerebral and peripheral), psychotropics, stimulants, antidiarrheal preparations, anti-anginal drugs, analgesics, anti-pyretics, hypnotics, sedatives, anti-emetics, growth promoters, anti-nauseants, anti-convulsants, neuro-muscular drugs, hyper and hypo glycemic agents, thyroid and anti-thyroid preparations, diuretics, cytotoxic compounds, anti-spasmodics, anti-arthritics, uterine relaxants, anti-obesity drugs, anthelmintics, hormones, vaccines, central nervous system agents not listed above, and mixtures thereof.

4. The composition according to claim 3 wherein said bioactive substance comprises crystalline ceftiofur free acid.

5. The composition according to claim 1 wherein said release is a sustained release.

6. A composition comprising crystalline ceftiofur free acid in a non-aqueous liquid carrier, wherein the water activity of said composition is between about 0.2 and about 0.5.

7. A method for producing a pharmaceutical composition comprising the steps of combining a non-aqueous liquid carrier with a bioactive substance and adjusting the carrier such that the water activity of the resulting composition is between about 0.2 and about 0.5.

8. The composition of any one of claims 1 to 6 for use in medical treatment.

9. The use of a composition of any one of claims 1 to 6 to prepare a medicament for treating or preventing a disease in a patient.

10. A method of treating or preventing a disease comprising administering to a patient in need of such treatment a composition of claim 1.

11. The method according to claim 10 wherein said disease is a bacterial infection.

12. The method according to claim 10 wherein said patient is a human.

13. The method according to claim 11 wherein said patient is a food animal or a companion animal.

14. The method according to claim 13 wherein said bioactive agent comprises crystalline ceftiofur free acid.

15. The method according to claim 14 wherein said method of treatment comprises a single administration of composition.

16. A composition comprising:
(a) one or more bioactive agents; and either
(b) one or more oxidizable oils;
(c) one or more oxidizable lipid components; or
(d) a mixture of one or more oxidizable oils and one or more oxidizable lipid components;
wherein said composition has been adjusted to have a water activity of between about 0.2 and about 0.5.
